(19) United States
(12) Patent Application Publication
(10) Pub. No.: US 2026/0107956 A1
(43) Pub. Date: Apr. 23, 2026

(54) RED SEAWEED FEED PRODUCTS AND METHODS FOR PROCESSING RED SEAWEED

(71) Applicant: CH4 Global, Inc., Henderson, NV (US)

(72) Inventor: Paul Joseph Russo, Henderson, NV (US)

(73) Assignee: CH4 Global, Inc., Henderson, NV (US)

(21) Appl. No.: 19/115,647

(22) PCT Filed: Sep. 28, 2023
(86) PCT No.: PCT/US2023/075390

§ 371 Date: Mar. 26, 2025

Related U.S. Application Data
(60) Provisional application No. 63/411,342, filed on Sep. 29, 2022.
(60) Provisional application No. 63/486,615, filed on Feb. 23, 2023.
(60) Provisional application No. 63/530,759, filed on Aug. 4, 2023.

(51) Int. Cl. A23K 10/30; A23K 10/33; A23K 20/105; A23K 20/158; A23K 30/20; A23K 40/10; A23K 50/10; A61K 9/00; A61K 31/02; A61K 36/04; A61P 1/14

(52) U.S. Cl. CPC A23K 10/30; A23K 10/33; A23K 20/105; A23K 20/158; A23K 30/20; A23K 40/10; A23K 50/10; A61K 9/0056; A61K 31/02; A61K 36/04; A61P 1/14; A61K 2236/15; A61K 2236/17

(57) ABSTRACT

According to an embodiment, a feed product may comprise from about 10 wt. % to about 95 wt. % of a red seaweed material; from about 6 wt. % to about 40 wt. % of total oil; and about 10 wt. % or less of water, based on the total weight of the feed product. Further embodiments include methods of making and methods of using the feed products disclosed herein.

### CROSS-REFERENCE TO RELATED APPLICATIONS

This application claims priority to U.S. Provisional Application No. 63/411,342 (Attorney Docket CBH0004MA), filed Sep. 29, 2022, and entitled “SEAWEED FEED PRODUCTS AND METHODS FOR PROCESSING SEAWEED.” and additionally claims priority to U.S. Provisional Application No. 63/486,615 (Attorney Docket: CBH0004MA1), filed Feb. 23, 2023, and entitled “SEAWEED FEED PRODUCTS AND METHODS FOR PROCESSING SEAWEED.” and additionally claims priority to U.S. Provisional Application No. 63/530,759 (Attorney Docket: CBH0006MA), filed Aug. 4, 2023, and entitled “RED SEAWEED FEED PRODUCTS AND METHODS FOR PROCESSING RED SEAWEED.” each of which is incorporated by reference it its entirety herein.

### BACKGROUND

### Field

The present specification generally relates to feed products containing red seaweed material and, more particularly, to feed products suitable for ruminants.

### Technical Background

With up to 1.5 billion domestic cattle worldwide, significant greenhouse gas (“GHG”) contribution globally is from cattle, sheep, and other ruminant production systems that are responsible for up to 20% of total global GHG emissions, primarily through emission of methane. Such methane emission is a byproduct of fermentation of feed organic matter in the rumen of the stomach of the unique digestive system of ruminant animals. Accordingly, an urgent need exists for methods and products which can reduce the methane emissions of ruminants.

### SUMMARY

According to one embodiment, a feed product may comprise from about 10 wt. % to about 95 wt. % red seaweed material; and from about 6 wt. % to about 40 wt. % of total oil; wherein the feed product comprises about 10 wt. % or less of water, based on the total weight of the feed product.

According to one embodiment, a method of making a feed product may comprise drying red seaweed to form a seaweed material; converting the seaweed material to particles; and combining the particles with from about 1 wt. % to about 35 wt. % of at least one added oil, by total weight of the feed product, thereby producing the feed product. The feed product may comprise about 10 wt. % or less of water, based on the total weight of the feed product.

According to one embodiment, a method for feeding a ruminant may comprise administering an amount of the feed product to the ruminant effective to reduce methane emissions in the ruminant.

It is to be understood that both the foregoing general description and the following detailed description describe various embodiments and are intended to provide an overview or framework for understanding the nature and character of the claimed subject matter.

### DETAILED DESCRIPTION

Reference will now be made in detail to embodiments of the feed products described herein, as well as method of processing and use. According to one or more embodiments, the feed products include red seaweed material and oil. As is described in detail herein, it has been discovered that administering seaweed material that includes one or more halogenated compounds, such as bromoform, to ruminants reduces the methane production of the ruminants. However, administering the seaweed material to ruminants may be difficult due to issues with mixing the seaweed with ruminant feed, palatability, inhalation hazards, food sorting by the ruminants, stability of the seaweed and its included halogenated compounds, stability of the feed product, or cost. Embodiments described herein meet these needs by providing feed products that comprise specific concentrations of red seaweed material, oil, and water. Further embodiments described herein are directed to methods of making and methods of administering the feed products.

As described herein, the embodiments are directed to feed products containing red seaweed material. A feed product, as described herein, refers to any material that is eaten (e.g., consumed and/or digested) by an animal, such as a ruminant, that includes the seaweed material or processed materials that are derived from the seaweed material. The feed products described herein, according to various embodiments, may be individually consumed by animals (for example the feed is consumed majorly without other feed materials), or may be consumed with other feeds (for example the feed is consumed in a mixture with other feed materials or “top dressed” with other feeds, such as forage). In some embodiments, the feed products described herein may constitute a relatively small amount of an animal's overall diet, and may be considered a supplement to another bulk feed. For example, the feeds described herein may be eaten by an animal along with other feeds such as, for example, forage (including, for example, grass or legume (for example, alfalfa) forage), silage, corn, soybeans, other seeds, oils, dietary supplements, or the like. For example, in some embodiments, the feed products described herein may be mixed with other feeds such as, for example corn and/or soy. In other examples, the animal may graze, or otherwise be provided any of a variety of forage, and be separately fed some amount of the feed products described herein. It is contemplated that the feed products described herein may be a portion of a feeding regimen, which may vary by ruminant breed and type, such as dairy cows, beef feed-lot cattle. “high end” cattle (e.g., Wagyu or other higher end cattle types), free range cattle, or the like, or may vary by feeding approaches (e.g., feed-lot or grazing systems or a combination of these). Each type of ruminant may have a specialized diet that includes the feed products along with other additives.

According to various embodiments, the feed products described herein may be consumed and/or digested by ruminants. As described herein and understood by those skilled in the art. “ruminants” may refer to herbivorous, hooved mammals (suborder Ruminantia and Tylopoda) that have a complex 3- or 4-chambered stomach. Ruminants include, without limitation, cattle, sheep, deer, goats, giraffes, camels, and llamas. The ruminants described herein may be domesticated, such as ruminants used for direct human food consumption, dairy production purposes, and/or recreation. In certain embodiments, the ruminants are cows. In some embodiments, the ruminants may be dairy cows, beef lot cattle, “high end” cattle such as Waygu, free range cattle, or others, or may vary by feeding approaches (e.g., feed-lot or grazing systems or a combination of these). In one embodiment, the ruminants are beef lot cattle. In another embodiment, the ruminants are dairy cattle. In another embodiment, the ruminants are free range cattle. In certain embodiments, the ruminants are sheep.

In embodiments described herein, the feed products include a red seaweed material containing one or more halogenated compounds (which are inherently present in the seaweed as harvested). As used herein, “red seaweed” means the family of seaweed known as Bonnemaisoniaceae and includes the genera, species, and variants (whether natural or synthetic) thereof. The Bonnemaisoniaceae family includes the Asparagopsis, Bonnemaisoniaceae, Delisea, Ptilonia, Leptophyllis, and Pleuroblepharidella genera. In one embodiment described herein, the seaweed material is a single genera or single species of the Bonnemaisoniaceae family, and in another embodiment it is a combination or two or more genera or species. In one embodiment herein, the seaweed material is Asparagopsis material. In one embodiment herein, the seaweed material is Asparagopsis taxiformis material. In another embodiment herein, the seaweed material is Asparagopsis armata material. In another embodiment herein, the seaweed material is a combination of Asparagopsis taxiformis material and Asparagopsis armata material. In any of the embodiments herein, the seaweed material is a processed material derived from a seaweed of the Bonnemaisoniaceae family, such as a processed material derived from Asparagopsis. In one embodiment herein, the seaweed material is dried, wherein such drying may be accomplished through methods described herein or in accordance with methods otherwise known to the ordinarily skilled artisan. As used herein, “dried” with reference to the seaweed material means that seaweed material is of a sufficiently dry state such that the feed product comprising the seaweed material comprises about 10% or less of water, based on the total weight of the feed product. Any of the red seaweed materials, including any materials processed therefrom, may be referred to herein simply as a seaweed material.

As described herein, “halogenated compound” refers to any chemical compound that includes a halogen (for example, fluorine, chlorine, bromine, iodine). As described herein, these halogenated compounds are generally present in the glands of some seaweeds. Throughout this disclosure, the description of a “halogenated compound” or “halogenated compounds” may refer to the one or more halogenated compounds that are present in the seaweed as harvested or following harvest and processing, such as specialized seaweed glands. It is contemplated herein that the feed product may comprise added halogenated compounds (compounds included in the feed product in addition to those already inherent in the seaweed material). In some embodiments, the halogenated compounds are organic, generally meaning that the halogen is bonded to a carbon molecular backbone, as would be understood by those skilled in the art. In certain embodiments, the halogenated compounds comprise halogens selected from the group consisting of fluorine, chlorine, bromine, and iodine.

A non-exhaustive listing of contemplated organic halogenated compounds includes bromoform; dibromo (iodo) methane; bromo (diiodo) methane; iodoform, dibromo (chloro) methane; bromo-chloro-iodomethane; dibromomethane; bromo (iodo) methane; diiodomethane; tetrabromomethane; acetyl iodide; 2-iodoethanol; 1-bromo-2-iodocthane; 2,2-dibromoacetaldehyde; 1-bromopropan-2-one; 1-iodopropan-2-one; 1,1-dibromopropan-2-one; 1-bromobutan-2-one; 1-bromo-3-iodopropan-2-one; 1,1,1-tribromopropan-2-one; 1,1-dibromo-1-chloropropan-2-one; 1,3-dibromobutan-2-one; 1,1-dibromo-3-iodopropan-2-one; 1,1,3,3-tetrabromopropan-2-one; 1,1,1,3,3,3-hexachloropropan-2-one; 1,1,3-tribromopropan-2-ol; 1,1,3,3-tetrabromoprop-1-enc; 1,1,3-tribromo-3-chloroprop-1-ene; 1,1-dibromo-3,3-dichloroprop-1-ene; 1,3,3-tribromo-1-iodoprop-1-ene; 3,3-dibromoprop-2-enal; 4,4-dibromobut-3-cn-2-one; 1,4,4-tribromobut-3-en-2-one; 1-iodo-4,4-dibromobut-3-en-2-one; 1,1,4,4-tetrabromobut-3-en-2-one; 1,4,4-tribromo-1-chlorobut-3-en-2-one; 1,1,4-tribromo-4-chlorobut-3-en-2-one; 1,1-dibromo-4,4-dichlorobut-3-en-2-one; 1,4-dibromo-1,4-dichlorobut-3-en-2-one; 2-chloroacetic acid; 2-broroacetic acid; 2-iodoacetic acid; 2,2-dichloroacetic acid; 2-bromo-2-chloroacetic acid; 2-iodo-2-chloroacetic acid; 2,2-dibromoacetic acid; 2-iodo-2-broroacetic acid; 2,2-diiodoacetic acid; 3-chloroprop-2-enoic acid; 2-chloroprop-2-enoic acid; 3-bromoprop-2-enoic acid; 3-iodoprop-2-enoic acid; 3-iodoprop-2-enoic acid; 3,3-dichloroprop-2-enoic acid; 2,3-dichloroprop-2-enoic acid; 3,3-dibromoprop-2-enoic acid; 2,3-dibromoprop-2-enoic acid; 3-iodo-3-dibromoprop-2-enoic acid; 2-iodo-3-bromoprop-2-enoic acid; 2-bromo-3-iodoprop-2-enoic acid; 3,3-diiiodoprop-2-enoic acid; 2,3-diiodoprop-2-enoic acid; 2,3,3-tribromoprop-2-enoic acid; 2,3-dibromo,3-iodoprop-2-enoic acid; 2-iodo-3,3-dibromoprop-2-enoic acid; bibromochloromethane, and bromochloromethane. At least bromoform, bibromochloromethane, and bromochloromethane and known to be constituents of some seaweed species such as Asparagopsis taxiformis and Asparagopsis armata.

In one embodiment, the halogenated compound includes bromine. Without limitation, of particular interest in the present embodiments is bromoform, which has been demonstrated to reduce methane emissions in ruminants. However, without being bound by any theory, it is believed that other halogenated compounds, besides bromoform, may also affect methane emission reduction in ruminants, and so the administration of these other compounds may be also beneficial.

The feed product comprises one or more oils. Some of the oil may be inherent in the seaweed material (i.e., present in the seaweed following harvest and drying procedures and before additional oils are added) and some of the oil may be mixed or otherwise combined with the seaweed material to form the feed product. Such mixed or otherwise combined oil is referred to herein as “added oil”. As used herein, the inherent oils and any added oils make up the total oil in the feed product. In embodiments, the feed product comprises from about 6 wt. % to about 40 wt. %, such as from about 7 wt. % to about 40 wt. %, about 8 wt. % to about 40 wt. %, about 10 wt. % to about 40 wt. %, about 12 wt. % to about 40 wt. %, about 15 wt. % to about 40 wt. %, about 20 wt. % to about 40 wt. %, about 25 wt. % to about 40 wt. %, about 6 wt. % to about 35 wt. %, about 7 wt. % to about 35 wt. %, about 8 wt. % to about 35 wt. %, about 10 wt. % to about 35 wt. %, about 12 wt. % to about 35 wt. %, about 15 wt. % to about 35 wt. %, about 20 wt. % to about 35 wt. %, about 6 wt. % to about 30 wt. %, about 7 wt. % to about 30 wt. %, about 8 wt. % to about 30 wt. %, about 10 wt. % to about 30 wt. %, about 12 wt. % to about 30 wt. %, about 15 wt. % to about 30 wt. %, about 20 wt. % to about 30 wt. %, or any subset thereof, of total oil, based on the total weight of the feed product. In one embodiment, the feed product comprises from about 22 wt. % to about 40 wt. %, or about 25 wt. % to about 40 wt. %, of total oil, based on the total weight of the feed product. In one embodiment, the feed product comprises from about 22 wt. % to about 35 wt. %, or about 25 wt. % to about 35 wt. %, of total oil, based on the total weight of the feed product. The quantity of total oil is measured according to the methods described in “Analysis of Fatty Acid Methyl Esters with High Accuracy and Reliability. IV Fats with Fatty Acids Containing Four or More Carbon Atoms,” JAOCS, Vol. 62, No. 10 (October 1985). It should be understood that, as needed, the person of ordinary skill in the art may make minor modifications to this method to measure the quantity of total oil in the feed product.

In some embodiments, the feed product may comprise up to about 5 wt. % of the oil inherent in the seaweed material (i.e., present in the seaweed following harvest and drying procedures and before additional oils are added). The composition of inherent oil may be the same or different as the added oil and may have been generated by the seaweed itself as it grew. The amount and composition of the oil may depend upon the species of red seaweed, the conditions under which it grew, was harvested, or otherwise processed, and the concentration of the seaweed in the feed product. In some embodiments, at least some of the oil inherent in the seaweed material comprises one or more fatty acid moieties having a carbon chain length of from 14-24 (i.e., C14-CH24). In some embodiments, at least some of the oil inherent in the seaweed material comprises one or more of butyric, caproic, caprylic, capric, lauric, myristic, myristoleic, pentadecanoic, palmitic, palmitoleic, margaric, heptadecenoic, stearic, octadecenoic, oleic, linoleic, alpha linoleic, gamma linoleic, dihomo-gamm-linoleic, octadecatrienoic, steridonic, arachidic, eicosenoic, cicosadienoic, eicosatrienoic, eicosatetraenoic, arachidonic, eicosapentaenoic, heneicosapentaenoic, docosanoic, erucic, docosenoic, docosatetraenoic, docosapentaenoic, docosahexaenoic, tetracosanoic, and tetracosanoic fatty acid moieties. In some embodiments, at least some of the oil inherent in the seaweed material comprises one or more of myristic, palmitic, palmitoleic, oleic, and linoleic fatty acid moieties. In some embodiments, the feed product may comprise 5 wt. % or less, about 4 wt. % or less, about 3 wt. % or less, about 2 wt. % or less, about 1.5 wt. % or less, or about 1 wt. % or less of the oil inherent in the seaweed material, based on the total weight of the feed product.

The feed product may further comprise an added oil. The added oil may be compositionally distinct from the oil inherent in the seaweed, or may be of the same or similar composition. As used herein, the term “oil” with reference to the added oil means any non-polar, hydrophobic substance which, in isolation, is typically liquid at ambient temperature and pressure. Oils may be derived from animals, plants, or petrochemicals, and typically have a high carbon and hydrogen content. In certain embodiments, the added oil is an edible oil. In certain embodiments, the added oil is derived from one or more plants (including those that are derived naturally and those that are made synthetically). The added oil may comprise an edible oil, such as a plant oil (for example, from botanical materials other than seaweed). The added oil may significantly reduce dust and odor may be masked, increasing safety and palatability. Additionally, the incorporation of the added oil may reduce the losses of active compounds such as bromoform or other organic halogenated compounds during harvesting, processing, transportation, storage, and administration of the feed product. Added oils may include, without limitation, almond oil, apricot oil, argan oil, avocado oil, brazil nut oil, cashew oil, canola oil, coconut oil, corn oil, cottonseed oil, flaxseed oil, grapefruit seed oil, grapeseed oil, hazelnut oil, hemp oil, lemon oil, linseed oil, macadamia oil, mustard oil, olive oil, orange oil, palm oil, palm kernel oil, peanut oil, pecan oil, pine nut oil, pistachio oil, pumpkin seed oil, rapeseed oil, rice bran oil, safflower oil, sesame oil, soybean oil, sunflower oil, vegetable oil (which may be a common reference to any of a variety of plant oils in combination, or a more specific combination of plant oils such as soybean oil, corn oil, or a combination of soybean and corn oils), walnut oil, or other plant oils, including any mixtures of any of the foregoing. In one embodiment, the added oil is canola oil, coconut oil, corn oil, cottonseed oil, flaxseed oil, grapeseed oil, olive oil, palm oil, palm kernel oil, peanut oil, rapeseed oil, safflower oil, sesame oil, soybean oil, sunflower oil, or vegetable oil, including any mixtures of any of the foregoing. In one embodiment, the added oil is canola oil, coconut oil, corn oil, cottonseed oil, flaxseed oil, olive oil, palm oil, peanut oil, rapeseed oil, safflower oil, sesame oil, soybean oil, sunflower oil, or vegetable oil, including any mixtures of any of the foregoing. In one embodiment, the added oil is canola oil, coconut oil, corn oil, olive oil, palm oil, peanut oil, rapeseed oil, safflower oil, soybean oil, including any mixtures of any of the foregoing. In one embodiment, the added oil is canola oil. In some embodiments, the added oil is one or more oils, two or more oils, or three or more oils. The feed product may comprise from about 1 wt. % to about 40 wt. %, from about 3 wt. % to about 40 wt. %, from about 5 wt. % to about 40 wt. %, from about 10 wt. % to about 40 wt. %, from about 15 wt. % to about 40 wt. %, from about 20 wt. % to about 40 wt. %. 1 wt. % to about 30 wt. %, from about 3 wt. % to about 30 wt. %, from about 5 wt. % to about 30 wt. %, from about 8 wt. % to about 30 wt. %, from about 10 wt. % to about 30 wt. %, from about 12 wt. % to about 30 wt. %, from about 5 wt. % to about 20 wt. %, from about 8 wt. % to about 20 wt. %, from about 10 wt. % to about 20 wt. %, from about 12 wt. % to about 20 wt. %, or any subset thereof, of the added oil, on the basis of the total weight of the feed product. In one embodiment, the feed product comprises from about 17 wt. % to about 35 wt. %, or about 20 wt. % to about 35 wt. %, or about 23 wt. % to about 35 wt. %, of added oil, or any subset thereof, based on the total weight of the feed product. In one embodiment, the feed product comprises from about 17 wt. % to about 30 wt. %, or about 20 wt. % to about 30 wt. %, or about 23 wt. % to about 30 wt. %, of added oil, or any subset thereof, based on the total weight of the feed product. The quantity added oil is measured according to the methods described in “Analysis of Fatty Acid Methyl Esters with High Accuracy and Reliability. IV Fats with Fatty Acids Containing Four or More Carbon Atoms.” JAOCS, Vol. 62, No. 10 (October 1985). It should be understood that, as needed, the person of ordinary skill in the art may make minor modifications to this method to measure the quantity of added oil in the feed product.

Without being limited by theory, it is believed that relatively higher total oil levels, including relatively higher added oil levels, provide a number of unexpected benefits relating to aspects such as bromoform stability and overall product efficacy. In addition, with these relatively higher total oil levels, the feed product may perform in a more efficacious and efficient manner when exposed to feeding conditions such as open feeding areas exposed to the environment, whether in beef or dairy lots, or in open grazing areas, such as high winds. As such, embodiments in which feed products comprise from about 22 wt. % to about 40 wt. %, or from about 25 wt. % to about 40 wt. %, of total oil, based on the total weight of the feed product, may be particularly beneficial. Embodiments in which feed products comprise from about 17 wt. % to about 35 wt. %, or from about 22 wt. % to about 35 wt. %, or from about 25 wt. % to about 35 wt. %, of added oil, based on the total weight of the feed product, may also be particularly beneficial.

The feed product may comprise relatively low amounts of water. In one embodiment, the feed product comprises seaweed material derived from red seaweed which has been dried in some manner following harvest, wherein the drying is in accordance with methods described herein or otherwise known to the ordinarily skilled artisan. The presence of excessive water may reduce the shelf life of the feed product by allowing bacterial growth, increase the weight and therefore shipping costs of the feed product, and degrade the halogenated compounds (e.g. bromoform) in the feed product. Accordingly, the feed product may comprise about 10 wt. % or less, about 8 wt. % or less, about 6 wt. % or less, about 4 wt. % or less, about 2 wt. % or less, or about 1 wt. % or less of water, based on the total weight of the feed product.

The feed product may have a water activity of about 0.85 or less at 25° C. Generally, water activity is a measure of how strongly bound water molecules are to the feed product. Even if water is present, if it is tightly bound to the feed product, it may not be available to support the growth of bacteria, yeasts, and mold. If the water activity is too high (i.e. not tightly bound), then the feed product may have sufficient moisture to grow bacteria, yeasts, and mold. Accordingly, feed products with sufficiently low water activities may be shelf stable and not require refrigeration or special handling. In embodiments, the feed product may have a water activity of about 0.6 or less, about 0.4 or less, or about 0.2 or less at 25° C. The water activity is measured according to ISO 18787:2017.

The feed product comprises a red seaweed material. The seaweed material may be derived from any genus of the Bonnemaisoniaceae seaweed family which include organic halogenated compounds, such as bromoform. Such seaweed species include, without limitation, Asparagopsis taxiformis or Asparagopsis Armata. However, it is contemplated that other species of red seaweed may include one or more organic halogenated compounds, or seaweed genetic variants may include one or more organic halogenated compounds, all of which are contemplated as applicable in the presently disclosed embodiments. As used herein, the seaweed material also includes any components or other materials derived from, processed from, or otherwise obtained from the seaweed material. The feed product may comprise from about 10 wt. % to about 95 wt. % of the seaweed material, based on the total weight of the feed product. As used herein, this is based on the dry weight of the seaweed material included in the feed product, although it will be understood that such dry weight of the seaweed material may necessarily contain some moisture content. As used herein, “dry weight” or “dried” with reference to the seaweed material means that seaweed material is of a sufficiently dry state such that the feed product comprising the seaweed material comprises about 10% or less of water, based on the total weight of the feed product. In embodiments, the feed product may comprise from about 10 wt. % to about 90 wt. %, about 10 wt. % to about 75 wt. %, about 10 wt. % to about 50 wt. %, about 10 wt. % to about 40 wt. %, about 20 wt. % to about 95 wt. %, about 20 wt. % to about 90 wt. %, about 20 wt. % to about 75 wt. %, about 20 wt. % to about 50 wt. %, about 20 wt. % to about 40 wt. %, about 30 wt. % to about 95 wt. %, about 30 wt. % to about 90 wt. %, about 30 wt. % to about 75 wt. %, about 30 wt. % to about 50 wt. %, about 30 wt. % to about 40 wt. %, about 40 wt. % to about 95 wt. %, about 40 wt. % to about 90 wt. %, about 40 wt. % to about 75 wt. %, about 40 wt. % to about 50 wt. %, or any subset thereof, of the seaweed material, based on the total weight of the feed product. The total weight of the feed product includes the weight of any water and/or oil present in the feed product, as well as any other optional components. In embodiments, the feed product may comprise at least about 25 wt. % or greater of the seaweed material, or from about 25 wt. % to about 45 wt. %, feed product, based on the total weight of the feed product.

To accomplish the goals of low water content, low water activity, and conversion of the seaweed material to particles, the seaweed may have been dried. As retrieved from the ocean or otherwise harvested from ponds or tanks, the seaweed may include as much as about 80 wt. % of water, by total weight of the retrieved or harvested seaweed material. Additionally, drying the seaweed may make it easier to convert the seaweed to particles. Drying the seaweed while frozen may provide additional stability benefits. Contemplated drying methods include, without limitation, freeze-drying, frozen sublimation drying (similar to freeze-drying but without the pressure change), and refractive window drying (in which IR energy is used to dry the product). In embodiments, the seaweed is freeze-dried seaweed material. Without being limited by theory, it is believed that freeze-dried seaweed material may have different structural characteristics than as-harvested seaweed or conventionally dried seaweed material.

The seaweed material in the feed product may be present as particles. As used herein “particles” refers to discrete fragments of the seaweed material which can be separated from other discrete fragments without substantial alteration in size or shape. For example, a particle of a given size may be able to pass through a sieve of that size without substantial alteration. In embodiments, about 80 wt. % or greater of the particles of seaweed material, based on the total weight of the particles, may have had a diameter, before being mixed with added oil, of from about 35 μm to about 420 μm. In embodiments, about 90 wt. % or greater of the particles, based on the total weight of the particles, may have had a diameter, before being mixed with added oil, of about 35 μm or greater, about 50 wt. % or greater of the particles may have had a particle size of about 160 μm or greater, and about 90 wt. % or greater of the particles may have had a particle size of about 420 μm or less.

In certain embodiments, the particles of the seaweed material are at least partially agglomerates of the seaweed material and added oil or other components of the feed product. Without being limited by theory, it is believed that cattle are adept at sorting food and may refuse to cat food particles which lack sufficient palatability or tactile preference. This sorting problem is believed to become markedly worse as the particles become larger, such as larger than 1 cm. Further, it is believed that extremely small particles, such as those having a diameter of less than 10 μm, may pose an inhalation hazard to animals and workers. The presence of such particles tends to require the use of expensive and time consuming personal protective equipment (PPE). Additionally, such small particles may tend to blow away, such as in a ruminant feeding environment, due to their extremely high surface area to mass ratio and the effects of static charge.

In certain embodiments, the feed product may comprise particles, including agglomerates, having a particle size distribution such that at least about 60 wt. %, at least about 70 wt. %, at least about 80 wt. %, at least about 90 wt. %, at least about 95%, or at least about 99 wt. % of the particles may have a diameter of from about 10 μm to about 1 cm, based on the total weight of the particles (including agglomerates) in the feed product. In certain embodiments, the feed product may comprise particles, including agglomerates, having a particle size distribution such that at least about 60 wt. %, at least about 70 wt. %, at least about 80 wt. %, at least about 90 wt. %, at least about 95%, or at least about 99 wt. % of the particles feed product may have a diameter of from about 10 μm to about 1 cm, from about 50 μm to about 1 cm, from about 100 μm to about 1 cm, from about 250 μm to about 1 cm, from about 500 μm to about 1 cm, from about 1000 μm to about 1 cm, from about 2500 μm to about 1 cm, from about 5000 μm to about 1 cm, from about 10 μm to about 8000 μm, from about 10 μm to about 5000 μm, from about 10 μm to about 2500 μm, from about 10 μm to about 1000 μm, or any subset thereof, based on the total weight of the particles in the feed product.

In certain embodiments, the feed product may comprise particles, including agglomerates, having a particle size distribution such that at least about 60 volume percent (vol. %), at least about 70 vol. %, at least about 80 vol. %, at least about 90 vol. %, at least about 95% vol. %, or at least about 99 vol. % of the particles may have a diameter of from about 10 μm to about 1 cm, based on the total volume of the particles (including agglomerates) in the feed product. In certain embodiments, the feed product may comprise particles, including agglomerates, having a particle size distribution such that at least about 60 vol. %, at least about 70 vol. %, at least about 80 vol. %, at least about 90 vol. %, at least about 95%, or at least about 99 vol. % of the particles feed product may have a diameter of from about 10 μm to about 1 cm, from about 50 μm to about 1 cm, from about 100 μm to about 1 cm, from about 250 μm to about 1 cm, from about 500 μm to about 1 cm, from about 1000 μm to about 1 cm, from about 2500 μm to about 1 cm, from about 5000 μm to about 1 cm, from about 10 μm to about 8000 μm, from about 10 μm to about 5000 μm, from about 10 μm to about 2500 μm, from about 10 μm to about 1000 μm, or any subset thereof, based on the total volume of the particles in the feed product.

For example, the feed product may comprise particles, including agglomerates of seaweed material and added oil, wherein at least about 95 wt. % of the particles may have a diameter of about 10 μm or greater, based on the total weight of the particles in the feed product. In embodiments, at least about 98 wt. %, at least about 99 wt. %, or at least about 99.9 wt. % may have a diameter of about 10 μm or greater, based on the total weight of the particles in the feed product.

For example, the feed product may comprise particles, including agglomerates of seaweed material and added oil, wherein at least about 95 vol. % of the particles may have a diameter of about 10 μm or greater, based on the total volume of the particles in the feed product. In embodiments, at least about 98 vol. %, at least about 99 vol. %, or at least about 99.9 vol. % may have a diameter of about 10 μm or greater, based on the total volume of the particles in the feed product.

As used herein, wherein the particles or agglomerates are not round, the diameter of the particles is the longest dimension. The size of the particles or agglomerates may be measured according to ISO 13320:2020, using a laser diffraction particle size analyzer, such as those available from Malvern Panalytical. Particles or agglomerates too large to be measured using a particle size analyzer may be measured using a microscopy technique, such as scanning electron microscopy or optical microscopy. Even larger particles or agglomerates may be measured using a sieve. Methodologies that may be used to measure particle and agglomerate size will be well-known to the ordinarily skilled artisan. Particle size may be expressed based on measurements of weight, volume, or count. Measurements based on volume of the particles and agglomerates are particularly provided for herein.

The feed product may be in the form of a powder. As used herein, a “powder” refers to a dry, bulk solid composed of many particles, such as agglomerates of seaweed material and added oil having a diameter from about 10 μm to about 1 cm, that may flow substantially freely when shaken, tilted, or otherwise moved. In a powder, the particles may move past one another to collectively take on the shape of their container. However, a powder may not include sufficient quantities of liquid to form a paste or gel.

According to additional embodiments, the feed product may further comprise molasses or other sugar material. In one embodiment herein, the molasses is dried molasses. Incorporation of molasses may significantly reduce dust and odor may be masked, increasing palatability. Without being bound by theory, it is believed that the incorporation of molasses may mitigate odor, improve palatability, and retain bromoform or other halogenated compounds. Molasses may be used in combination with added oil. The quantity of molasses or other sugar material used may be high enough to be effective yet low enough that the feed product is still a powder. In one embodiment, the feed product comprises from about 1% to about 50% molasses, or from about 2% to about 40% molasses, or from about 5% to about 30% molasses, or from about 10% to about 25% molasses, or from 10% to 20% molasses, or from 12% to 18% molasses, or from 14% to 16% molasses, based on the total weight of the feed product. It should be understood that the feed product may comprise both molasses and oil. In certain embodiments, the feed product may comprise from 10% to 20% molasses, from 10% to 20% added oil, and from 60% to 80% seaweed material, based on the total weight of the feed product. In any of the foregoing embodiments, the molasses may be dried molasses of a composition that will be well-known to the ordinarily skilled artisan.

As described herein, the feed product may include a binding agent. The binding agent may bind to one or more halogenated compounds, such as bromoform, resulting in one or more bound halogenated compounds. The material that includes the halogenated compound bound with the binding agent is sometimes referred to herein as the bound halogenated compound material, where the one or more halogenated compounds are bound to a binding agent. Without being limited by theory, it is believed that a bound halogenated compound may be more stable than an unbound halogenated compound during transport, storage, and processing of the feed product or the ingredients of the feed product.

In one or more embodiments, the binding agent may be any material (including mixtures of chemical compounds) that is capable of binding with the desired halogenated compound or compounds. As described herein, halogenated compound that is “bound” with the binding agent refers to halogenated compound that is chemically bonded to the binding agent. Various chemical bonds are contemplated, such as chemical association (e.g., a complex formed by two or more compounds), hydrogen bonding, covalent bonding, ionic bonding, van der Waals bonding, or polar covalent bonding. Generally, the bonding of these materials, such as cyclic oligosaccharides, will be understood by those skilled in the art. For example, a cyclodextrin may from a complex, or be otherwise associated with, the one or more halogenated compounds contemplated herein. In one non-limiting embodiment, one or more cyclodextrins are bound with bromoform, forming a solid precipitate, by forming a chemical complex or association.

As described herein, the binding agent included in the feed products described herein may be bound or unbound with one or more halogenated compounds. For example, a portion of the binding agent may be bound with the halogenated compound or compounds and a portion may not be bound with the halogenated compound or compounds. For example, excess binding agent may be present as compared with the stoichiometric bound equivalent of halogenated compound so that all halogenated compound is bound.

According to one or more embodiments, the feed product may comprise about 0.01 wt. % or greater of the binding agent (including bound and non-bound binding agent). In additional embodiments, the feed product may comprise less than about 0.01 wt. %, about 0.02 wt. % or greater, about 0.03 wt. % or greater, about 0.04 wt. % or greater, about 0.05 wt. % or greater, about 0.1 wt. % or greater, about 0.2 wt. % or greater, about 0.3 wt. % or greater, about 0.4 wt. % or greater, about 0.5 wt. % or greater, about 0.6 wt. % or greater, about 0.7 wt. % or greater, about 0.8 wt. % or greater, about 0.9 wt. % or greater, or even about 1 wt. % or greater of the binding agent, based on the total weight of the feed product. As described herein, the amount of binding agent does not include the halogenated compound that may be bound with the binding agent. By way of example, a mixture of about 1 wt. % cyclodextrin (1 g per 100 g seaweed material) may bind about 200 mg bromoform, or about 0.2% in unharvested seaweed.

According to various embodiments, the binding agent may be a naturally occurring or synthetic material or combination of materials with limited or no pharmaceutical activity. For example, the binding agent may comprise materials recognized by those skilled in the art to be harmless to animals when consumed and may be naturally occurring or synthetic materials. In some embodiments, the feed products described herein do not include any active pharmaceutical ingredients or bioactive materials aside from those that may be present in unharvested seaweed. For example, and without limitation, some cyclic oligosaccharides, such as cyclodextrins, are considered to be substantially or wholly harmless to ruminants and other animals when consumed.

According to one or more embodiments, the binding agents may be organic binding agents or inorganic binding agents, as are discussed herein. Organic binding agents generally include a carbon backbone structure, whereas inorganic binding agents do not, as would be understood by those skilled in the art.

In some embodiments, the organic binding agent may comprise one or more cyclic oligosaccharides. As used herein, the term “cyclic oligosaccharide” means a cyclic structure comprising six or more saccharide units. Some exemplary embodiments for use herein are cyclic oligosaccharides having six, seven, or eight saccharide units and combinations thereof. It is common in the art to abbreviate six, seven, and eight membered cyclic oligosaccharides to a. B. and y, respectively.

The cyclic oligosaccharide of the compositions used for the present embodiments may comprise any suitable saccharide or mixtures of saccharides. Examples of suitable saccharides include, but are not limited to, glucose, fructose, mannose, galactose, maltose, and combinations thereof. In one or more embodiments, the cyclic oligosaccharides for use herein are α-cyclodextrins. β-cyclodextrins, or combinations thereof. In one or more embodiments, the cyclic oligosaccharides for use herein are α-cyclodextrins. In one or more embodiments, the cyclic oligosaccharides for use herein are β-cyclodextrins.

The cyclic oligosaccharides, or mixture of cyclic oligosaccharides, utilized in the embodiments described herein may be substituted by any suitable substituent or mixture of substituents. Herein the use of the term “mixture of substituents” means that two or more different suitable substituents can be substituted onto a cyclic oligosaccharide. The derivatives of cyclodextrins may consist mainly of molecules wherein some of the hydroxyl groups have been substituted. Suitable substituents include, but are not limited to, alkyl groups; hydroxyalkyl groups; dihydroxyalkyl groups; (hydroxyalkyl)alkylenyl bridging groups such as cyclodextrin glycerol ethers; aryl groups; maltosyl groups; allyl groups; benzyl groups; alkanoyl groups; cationic cyclodextrins such as those containing 2-hydroxy-3-(dimethylamino) propyl ether; quaternary ammonium groups; anionic cyclodextrins such as carboxyalkyl groups, sulphobutylether groups, sulphate groups, and succinylates; amphoteric cyclodextrins; and combinations thereof.

The substituents may be saturated or unsaturated, straight or branched chain. Some substituents include saturated and straight chain alkyl groups, hydroxyalkyl groups, and combinations thereof. Some alkyl and hydroxyalkyl substituents are selected from C1-C8 alkyl or hydroxyalkyl groups or combinations thereof. In some embodiments, alkyl and hydroxyalkyl substituents are selected from C₁-C₆ alkyl or hydroxyalkyl groups, or combinations thereof. In additional embodiments, alkyl and hydroxyalkyl substituents are selected from C₁-C₄ alkyl or hydroxyalkyl groups and combinations thereof. Embodiments of alkyl and hydroxyalkyl substituents include propyl, ethyl and methyl.

In one or more embodiments, cyclic oligosaccharides for use in the presently disclosed embodiments are unsubstituted, or are substituted by only saturated straight chain alkyl, or hydroxyalkyl substituents. Therefore, some examples of cyclic oligosaccharides for use herein are α-cyclodextrin. β-cyclodextrin, methyl-α-cyclodextrin, methyl-β-cyclodextrin, hydroxypropyl-α-cyclodextrin and hydroxypropyl-β-cyclodextrin. One or more of these compounds are available from Wacker-Chemie Gmbh Hanns-Seidel-Platz 4, Munchen, DE under the tradename Alpha W6 M and Beta W7 M respectively.

According to additional embodiments, the organic binding agent may comprise one or more amphiphilic components that combine to form aggregate structures in aqueous systems, said aggregate structures possessing a lipophilic interior in some embodiments. Non-limiting examples of amphiphilic components include ethoxylated castor oil and ethoxylated hydrogenated castor oil. In additional embodiments, the organic binding agent may comprise one or more amphiphilic multi-arm star-block copolymers such as those described by Ternat, et al. (Macromol. Chem. Phys. 208:131 2007). In yet additional embodiments, the organic binding agent may also comprise one or more polymeric emulsifiers possessing a lipophilic portion and a hydrophilic portion. Non-limiting examples of such polymeric emulsifiers include Acrylates/C10-30 Alkyl Acrylate Crosspolymers available under the tradename PEMULEN by Lubrizol Advanced Materials, Inc. (Cleveland, Ohio, USA).

In additional embodiments, the binding agents may be inorganic binding agents such as, without limitation, molecular sieves such as zeolites. Zeolites may be crystalline aluminosilicates formed by corner sharing [SiO4]⁴⁻or [AlO₄]⁵⁻ tetrahedra, which possess periodic one-to-three-dimensional frameworks, unique pore structure, and fine physical and chemical stabilities.

According to one or more embodiments described herein, the feed product may additionally comprise one or more quality extenders. In one embodiment, the feed products may comprise one or more quality extenders to assist with ensuring an appropriate level of halogenated compound relative to the total weight of the feed product. For example, the quality extenders described herein may further comprise one or more grains such as barley, sorghum, oats, wheat, corn or other similar grains that may be fed to cattle or other ruminants; grasses/silage materials that are whole, ground or pelleted including lucerne, corn silage, straw, or hay; cellulosic waste streams that are whole or ground including cotton seed, almond hulls, spent grains, citrus peels; or combinations of these. The one or more quality extenders may be whole, ground, steamed, processed to a flour, or otherwise processed to result in a desired form. In one embodiment, the feed products comprise barley flour. In another embodiment, the feed products comprise wheat flour. In another embodiments, the feed products comprise barley flour and wheat flour. In some embodiments, the feed products described herein may comprise from about 15 wt. % to about 85 wt. %, from about 15 wt. % to about 75 wt. %, from about 15 wt. % to about 65 wt. %, from about 15 wt. % to about 55 wt. %, from about 15 wt. % to about 45 wt. %, from about 15 wt. % to about 35 wt. %, from about 25 wt. % to about 85 wt. %, from about 25 wt. % to about 75 wt. %, from about 25 wt. % to about 65 wt. %, from about 25 wt. % to about 55 wt. %, from about 35 wt. % to about 85 wt. %, from about 35 wt. % to about 75 wt. %, from about 35 wt. % to about 65 wt. %, from about 35 wt. % to about 55 wt. %, from about 45 wt. % to about 85 wt. %, from about 45 wt. % to about 75 wt. %, from about 45 wt. % to about 65 wt. %, from about 45 wt. % to about 55 wt. %, or any subset thereof, of the quality extenders, based on the total weight of the feed product.

In additional embodiments, the feed products described herein may further comprise one or more flow control agents such as silica (including, for example, fumed silica), tri calcium phosphate, or the like to avoid clumpiness, allow for material flow in bulk situations. In embodiments, the feed products described herein may comprise from about 0 wt. % to about 2 wt. %, from about 0 wt. % to about 1.5 wt. %, from about 0 wt. % to about 1 wt. %, from about 0.5 wt. % to about 2 wt. %, from about 0.5 wt. % to about 1.5 wt. %, from about 0.5 wt. % to about 1.5 wt. %, from about 0.75 wt. % to about 1.25 wt. %, or any subset thereof, of the flow control agents, based on the total weight of the feed product. One or more flow control agents may be particularly useful in feed products having relatively higher total oil or added oil levels. In such embodiments, feed products comprising from about 1.25 wt. % to about 2 wt. % or from about 1.5 wt. % to about 2 wt. %, of the flow control agents, based on the total weight of the feed product, may be particularly beneficial. In additional embodiments, the feed products described herein may further comprise other stability enhancers such as antioxidants (e.g., viatmin E) etc. to avoid any oxidation stability concerns. In additional embodiments, the feed products described herein may further comprise added vitamins, minerals, and other nutrients. In further embodiments, the feed products described herein may incorporate active pharmaceutical ingredients.

The feed product may comprise from about 0.2 wt. % to about 2 wt. % of one or more halogenated compounds, such as bromoform, of the halogenated compounds, based on the total weight of the feed product. In embodiments, the feed product may comprise from about 0.2 wt. % to about 1.8 wt. %, about 0.2 wt. % to about 1.6 wt. %, about 0.2 wt. % to about 1.4 wt. %, about 0.2 wt. % to about 1.2 wt. %, about 0.2 wt. % to about 1 wt. %, about 0.2 wt. % to about 0.8 wt. %, about 0.2 wt. % to about 0.7 wt. %, about 0.4 wt. % to about 2 wt. %, about 0.5 wt. % to about 2 wt. %, about 0.6 wt. % to about 2 wt. %, about 0.8 wt. % to about 2 wt. %, about 1 wt. % to about 2 wt. %, about 1.2 wt. % to about 2 wt. %, about 1.4 wt. % to about 2 wt. %, about 1.6 wt. % to about 2 wt. %, about 1.8 wt. % to about 2 wt. %, about 0.4 wt. % to about 1 wt. %, about 0.5 wt. % to about 1 wt. %, about 0.2 wt. % to about 0.8 wt. %, about 0.2 wt. % to about 0.7 wt. %, about 0.4 wt. % to about 0.8 wt. %, about 0.5 wt. % to about 0.7 wt. %, or any subset thereof, of the halogenated compounds, based on the total weight of the feed product.

According to additional embodiments, the present disclosure is directed to methods for feeding ruminants. According to such methods, an amount of feed product that is effective to reduce methane emissions in the ruminant may be administered to the ruminant. As described herein, administering the feed product may include a variety of steps, such as providing the ruminant with the feed product and allowing the ruminant to eat and digest the feed product. For example, the feed product could be mixed with other feeds or could be supplied individually to the ruminant. Administration of the feed products disclosed herein to animals can be, according to various embodiments, on a continuous basis (e.g., eaten daily with normal food rations) or at particular spaced apart times (e.g., about once a week or once a month).

In embodiments, in addition to the feed product, the ruminant may also be administered forage, grain, or combinations thereof. The feed product may be mixed with the forage, grain, or both in a mixing device. Then, the combined feed product and forage/grain may be administered to the ruminant. In embodiments, the ruminant may be administered the feed product alone as a treat, and the forage, grain, or combination thereof may be administered separately.

According to one or more embodiments, a method of making a feed product may comprise drying a red seaweed to form a seaweed material; converting the seaweed material to particles; and mixing the particles with from about 1 wt. % to about 35 wt. % of at least one added oil, on the basis of the total weight of the feed product, thereby producing the feed product. In some embodiments, the method may comprise mixing the particles with from about 1 wt. % to about 40 wt. %, from about 3 wt. % to about 40 wt. %, from about 5 wt. % to about 40 wt. %, from about 10 wt. % to about 40 wt. %, from about 15 wt. % to about 40 wt. %, from about 20 wt. % to about 40 wt. %, 1 wt. % to about 30 wt. %, from about 3 wt. % to about 30 wt. %, from about 5 wt. % to about 30 wt. %, from about 8 wt. % to about 30 wt. %, from about 10 wt. % to about 30 wt. %, from about 12 wt. % to about 30 wt. %, from about 5 wt. % to about 20 wt. %, from about 8 wt. % to about 20 wt. %, from about 10 wt. % to about 20 wt. %, from about 12 wt. % to about 20 wt. %, or any subset thereof, of the added oil, based on the total weight of the feed product. The feed product may comprise about 10 wt. % of water or less, based on the total weight of the feed product. In embodiments, the feed product may comprise about 8 wt. % or less, about 6 wt. % or less, about 4 wt. % or less, about 2 wt. % or less, or about 1 wt. % or less of water, based on the total weight of the feed product.

In one embodiment herein, it may be advantageous to provide a feed product comprising relatively low levels of iodine. In one embodiment, the feed product comprises about 0.5 wt. % or less, about 0.25 wt. % or less, about 0.1 wt. % or less, or about 0.05 wt. % or less of iodine, based on the total weight of the feed product. In one embodiment, the feed product comprises from about 0.01 wt. % to about 0.5 wt. %, from about 0.01 wt. % to about 0.25 wt. %, from about 0.05 wt. % to about 0.25 wt. %, or from about 0.1 wt. % to about 0.25 wt. % of iodine, or any subset thereof, based on the total weight of the feed product. The ordinarily skilled artisan will understand that such levels of iodine are measured based on an analysis of iodide content present from any of a variety of the total iodine-containing compounds that may be present.

According to one or more embodiments, a harvested red seaweed may be supplied. In some embodiments, the harvested seaweed may be supplied by harvesting a precursor seaweed. As described herein, the precursor seaweed is the seaweed that forms, following any of a variety of processing steps (e.g., cutting, packaging, etc.), the seaweed material of the feed products described herein. As discussed herein, harvesting, may refer generally to gathering the seaweed crop, such as by cutting or other mechanical means, and removing the seaweed from its growing habitat. In other embodiments, the harvested seaweed may be supplied by another party, and the following steps are equally applicable.

As described herein, it is contemplated that additional processing steps may occur between harvesting and/or supplying of a harvested seaweed and initial contact with the aqueous solution. For example, in some embodiments, the harvested seaweed may be transported by boat from its harvesting site prior to contact with the aqueous solution. In additional embodiments, the harvested seaweed may be physically altered, such as by cutting, chopping, or the like, prior to the drying, converting, and mixing steps.

The red seaweed may be dried to form a seaweed material. Drying the seaweed may help to preserve the halogenated compounds in the seaweed material and may help to preserve the seaweed material, and the feed product, by lowering the water activity. Additionally, in some embodiments where the seaweed is dried before being converted to particles, it may be easier to convert the seaweed to the particles. Contemplated drying methods include, without limitation, freeze-drying, frozen sublimation drying (similar to freeze-drying but without the pressure change), and refractive window drying (where IR energy is used to dry the product). Upon drying, the seaweed material may contain some levels of moisture as will be well-understood by the ordinarily skilled artisan.

The seaweed may be converted to particles. In embodiments, the seaweed material may be converted to particles by crushing, grinding, milling, or similar processes.

The seaweed material may be mixed with, or otherwise combined with, at least one added oil. In certain embodiments, the seaweed material may be mixed with the at least one added oil by spraying the seaweed material with the added oil, by mixing the seaweed material and added oil in a mixer, by coextruding the seaweed material and added oil, or any of a combination of these methods or other methods that will be well-understood by the ordinarily skilled artisan.

The seaweed material may be combined with at least one of a binding agent, a flow control agent, molasses, or a quality extender. In embodiments, the seaweed material may be mixed or otherwise combined with at least one of a binding agent, a flow control agent, molasses, or a quality extender in a mixer, extruder, or other similar mixing device.

In one or more embodiments, the seaweed material in the feed products may also be processed to reduce the content of water-soluble salts, especially halide salts. For example, reduction of these materials may lead to improved palatability for a ruminant. In some embodiments, the seaweed feed material may have improved palatability to a cow, a sheep, or other ruminants, as compared to freshly harvested seaweed.

In one or more embodiments, a method of feeding a ruminant animal the feed product of the present invention is provided herein. In one or more embodiments, a method of reducing methane emissions in a ruminant animal comprising feeding the ruminant animal the feed product of the present invention is provided herein. In one embodiment, the feed product is administered to the ruminant animal for about 10 days or more, or about 20 days or more, or about 30 days or more, or about 60 days or more, or about 120 days or more, or 150 about days or more, or 180 about days or more. In one embodiment, the feed product is administered in conjunction with other feed as described hereinabove to form a total mixed ration. In one embodiment, the method of feeding the ruminant animal or reducing methane emissions in the ruminant animal comprises feeding about 0.1% or more, about 0.25% or more, about 0.5% or more, about 0.75% or more, or about 1% or more of the feed product, by weight to the total mixed ration feed to the ruminant animal, on a daily basis. The ordinarily skilled artisan will understand that feeding ruminant animals in beef feed lots, dairy lots, or in free range environments may be variable due to the difficulty in feeding precise levels of total mixed ration to the animal. Feeding instructions, such as accompanying packaging or other printed matter, may provide the foregoing administration information as a guideline.

In additional embodiments, the feed product herein may be administered to the ruminant through use of a bolus or a lick. In such embodiments, the bolus or lick may comprise, consist, or consist essentially of (i.e., 99 wt. % or greater) the feed product and other conventional materials supplied to ruminants via lick or bolus. In some embodiments, other additives may be present in the bolus or lick, such as conventionally known substances that are present in known boluses and licks, as described herein.

As described herein, a bolus refers to a type of oral supplement that is commonly used for ruminants, such as cattle, sheep, and goats. The bolus may be a relatively large, capsule-like tablet that is designed to release its contents slowly over a period of time. Boluses can be used for various purposes, such as to deliver minerals, vitamins, or medications to the animal, or to treat specific conditions such as parasite infestations. Typical ruminant boluses may be made up of an outer layer of a hard, slow-dissolving material that surrounds a core of the active ingredients. The bolus may be designed to remain in the animal's rumen (the first compartment of its four-chambered stomach) for several weeks or months, slowly releasing the contents as the outer layer dissolves. The slow release of the bolus may ensure that the animal receives a relatively consistent dose of the active ingredients (such as bromoform) over a prolonged period of time.

As describe herein, a lick refers to a mixture of materials such as salts, minerals, and sometimes other ingredients (such as bromoform) that are made into a concentrated block or spread on a flat surface. These licks are made available to ruminants, such as cattle, sheep, and goats, as a way to supplement their diet with essential minerals that may not be present in their normal feed. For example, a common lick for ruminants can be made with a mixture of salt, minerals such as magnesium, calcium, and phosphorus, and sometimes molasses or other ingredients to make it more palatable. Licks contemplated herein may further include halogenated compounds such as bound bromoform). The lick may then formed into a block or spread on a flat surface, such as a metal or plastic plate, and made available to the animals.

Without being limited by theory, it is believed that the incorporation of the feed product into a bolus may contribute to controlled release of the halogenated material. Additionally, it is believed that the incorporation of the feed product into a lick may contribute to improved stability of halogenated materials, such as bromoform.

Without limitation, contemplated embodiments of boluses include those that may have a lifetime of about 30 days and deliver about 300 mg of bromoform per day. Such a bolus may utilize about 9 grams of bromoform, where the total weight of the bolus may be about 100 grams.

### Non-Limiting Aspects

According to a first aspect, a feed product may comprise: from about 10 wt. % to about 95 wt. % red seaweed material and from about 6 wt. % to about 40 wt. % of total oil; wherein the feed product comprises about 10 wt. % or less of water, based on the total weight of the feed product.

According to a second aspect, in conjunction with the first aspect, the seaweed material may comprise one or more species of Asparagopsis, such as Asparagopsis taxiformis and/or Asparagopsis armata.

According to a third aspect, in conjunction with the first or second aspect, the feed product may comprise from about 6 wt. % to about 30 wt. % of total oil, based on the total weight of the feed product.

According to a fourth aspect, in conjunction with any one of aspects 1 to 3, the feed product may comprise from about 10 wt. % to about 30 wt. % of total oil, based on the total weight of the feed product.

According to a fifth aspect, in conjunction with any one of aspects 1 to 4, the feed product may comprise about 25 wt. % or greater of seaweed material, based on the total weight of the feed product.

According to a sixth aspect, in conjunction with any one of aspects 1 to 5, wherein the feed product may comprise particles.

According to a seventh aspect, in conjunction with aspect 6, the particles of the feed product may comprise agglomerations of seaweed material particles and added oil.

According to an eighth aspect, in conjunction with aspect 7, the particles, including the agglomerations of seaweed material particles and added oil, may have a diameter of from 10 μm to 1 cm.

According to a ninth aspect, in conjunction with any one of aspects 1 to 8, at least about 90 vol. % or greater of the particles have a diameter of about 10 μm or greater, based on the total weight of all particles in the feed product.

According to a tenth aspect, in conjunction with any one of aspects 1 to 9, wherein the seaweed material may comprise Asparagopsis taxiformis. Asparagopsis armata, or both.

According to an eleventh aspect, in conjunction with any one of aspects 1-10, wherein the total oil may comprise an added oil and oils inherent in the seaweed material, wherein the added oil comprises one or more of canola oil, soybean oil, grape seed oil, coconut oil, palm oil, rapeseed oil, sunflower seed oil, peanut oil, cottonseed oil, palm oil, or olive oil.

According to a twelfth aspect, in conjunction with any one of aspects 1 to 11, the feed product may further comprise dried molasses.

According to a thirteenth aspect, in conjunction with any one of aspects 1 to 12, the feed product may further comprise a flow control agent.

According to a fourteenth aspect, in conjunction with any one of aspects 1 to 13, the feed product may further comprise a binding agent.

According to a fifteenth aspect, in conjunction with aspect 14, the binding agent may comprise α-cyclodextrin. β-cyclodextrin, one or more zeolites, or combinations thereof.

According to a sixteenth aspect, in conjunction with any one of aspects 1 to 15, the feed product may further comprise one or more quality extenders.

According to a seventeenth aspect, in conjunction with any one of aspects 1 to 16, the feed product may comprise about 5 wt. % or less of water, based on the total weight of the feed product.

According to an eighteenth aspect, in conjunction with any one of aspects 1 to 17, the feed product may have a water activity of about 0.6 or less at 25° C.

According to a nineteenth aspect, in conjunction with any one of aspects 1 to 18, the feed product may have a water activity of about 0.2 or less at 25° C.

According to a twentieth aspect, in conjunction with any one of aspects 1 to 19, the seaweed material may be freeze-dried seaweed.

According to a twenty-first aspect, in conjunction with any one of aspects 1 to 20, the feed product may comprise from 0.2 wt. % to 2 wt. % of halogenated compounds, based on the total weight of the feed product.

According to a twenty-second aspect, in conjunction with aspect claim 21, the halogenated compounds may comprise bromoform.

According to a twenty-third aspect, in conjunction with any one of aspects 1 to 22, a method for feeding a ruminant may comprise administering an amount of the feed product to the ruminant effective to reduce methane emission in the ruminant.

According to a twenty-fourth aspect, in conjunction with aspect 23 the ruminant may be a cow or a sheep.

According to a twenty-fifth aspect, in conjunction with either one of aspects 23 or 24, the method may further comprise administering forage, grain, or combinations thereof, to the ruminant.

According to a twenty-fifth aspect, in conjunction with any one of aspects 23 to 25, the method may further comprise mixing the feed product and a ruminant feed in a mixing device, prior to administering the feed product to the ruminant.

According to a twenty-seventh aspect, alone or in conjunction with any one of aspects 1 to 26, a method of making a feed product may comprise: drying a red seaweed to form a seaweed material; converting the seaweed material to particles; and combining the particles with about 1 wt. % to about 35 wt. % of at least one added oil, thereby producing the feed product, wherein the feed product comprises about 10 wt. % or less of water, on the basis of the total weight of the feed product.

According to a twenty-eighth aspect, in conjunction with aspect 27, drying the seaweed may comprise freeze-drying.

### EXAMPLES

The various aspects of the present disclosure will be further clarified by the following examples. The examples are illustrative in nature and should not be understood to limit the subject matter of the present disclosure.

### Comparative Example 1 (CE-1)

In a first comparative example, an Asparagopsis seaweed (New Zealand wild harvested Asparagopsis armata) was freeze dried and ground to form a seaweed material. The freeze dried and ground seaweed material was then fed into a Malvern Mastersizer 3000 laser analyzer to determine particle size and other properties. The freeze-dried, ground seaweed material had a specific surface area of 1203 m²/kg, a total and inherent oil content of from 0.6 wt. % to 1.5 wt. %, a moisture content of from 4 wt. % to 5 wt. %, a water activity of from 0.12 to 0.14, and a bromoform (BF) content of from 1 mg BF/g seaweed to 2 mg BF/g seaweed. It is noted that this relatively low level of bromoform content may not be suitable for feed to animals and was used as a representative material for these tests. Dimensional properties of the examples are shown below in Table 1. The percentiles refer to the volume percentages. For example, 10 vol. % of the particles in CE-1 have a particle size of less than 38.1 μm.

### Comparative Example 2 (CE-2)

The sample of CE-1 was mixed with 3 wt. % of added oil to form sample CE-2. Sample CE-2 was then fed to the Malvern laser analyzer and results are shown in Table 1 below.

### Example A (EX-A)

The sample of CE-1 was mixed with 5 wt. % of added oil to form sample EX-A. Sample EX-A was then fed to the Malvern laser analyzer and results are shown in Table 1 below.

### Example B (EX-B)

The sample of CE-1 was mixed with 7 wt. % of added oil to form sample EX-B. Sample EX-B was then fed to the Malvern laser analyzer and results are shown in Table 1 below.

### Example C (EX-C)

The sample of CE-1 was mixed with 10 wt. % of added oil to form sample EX-C. Sample EX-C was fed to the Malvern laser analyzer but the particles were too large to assess by the Malvern. However, sample EX-C was still a powder upon visual inspection. The particles may be measured using a sieve.

TABLE 1

| | CE-1 | CE-2 | EX-A | EX-B |
|---|---|---|---|---|
| D10 (μm) | 38.1 | 40 | 45 | 64 |
| D50 (μm) | 166 | 177 | 213 | 339 |
| D80 (μm) | 321 | 438 | 503 | 785 |
| D84 (μm) | 351 | 516 | 584 | 930 |
| D90 (μm) | 411 | 742 | 792 | 1284 |
| D99 (μm) | 516 | 2174 | 2079 | 2632 |
| wt. % under 10 μm | 2.3 | 1.44 | 0.51 | 0.06 |
| Mean (μm) | 174 | 318.98 | 346 | 460 |

As can be seen from Table 1, as the quantity of added oil increases, the particle sizes increase. In particular, the quantity of particles sized at 10 μm decreases dramatically with 5 wt. % of added oil. This decrease accelerates with the addition of at least 7 wt. % of added oil.

### Example D

A feed product of the present invention is manufactured and comprises Asparagopsis seaweed, wheat flour, dried molasses, and canola oil. The feed product of Example D has the following characteristics as indicated in Table 2.

TABLE 2

| CATEGORY | NUTRIENT | AMOUNT | |
|---|---|---|---|
| Feed | Neutral detergent fiber (min) | 18 | wt. % |
| characteristics | Crude protein (min) | 13 | wt. % |
| | Crude fat (min) | 15 | wt. % |
| | Ash (max) | 40 | wt. % |
| Key | Iodine | 0.16 wt. % | |
| components | | (max) | |
| | Bromoform | 0.6 wt. % | |
| | | (min) | |
| Micronutrients | Calcium | 1 | wt. % |
| | Magnesium | 0.5 | wt. % |
| | Sodium | 5.5 | wt. % |
| | Sulfur | 2 | wt. % |
| Trace elements | Iron | 300 | ppm |
| Other | Present at least than 2 wt. % of the feed | | |
| nutrients | product: chlorine, potassium, | | |
| | phosphorous, manganese, zinc, selenium, | | |
| | cobalt, copper, Vitamin A, Vitamin D | | |

### Example E

The feed product of Example EX-A. EX-B. EX-C. or D is manufactured in quantities sufficient to feed at least 50 head of beef lot or dairy cattle. On a daily basis for 150 days, each head of cattle is administered about 50 grams of the feed product per 10 kilograms of total mixed ration (i.e., about 0.5% of the feed product, based on the total weight of total mixed ration). The total mixed ration is based on the preferences of feeding lot/farm ordinary customs and procedures and may include silage and grain. The feed product is top-dressed or otherwise included as a supplement to the total mixed ration daily diet. Beef or dairy products resulting from the cattle fed in accordance with this example are accompanied by one or more of the following statements:
Up to 90% reduction of enteric methane emissions from beef feedlot cattle
Up to 90% reduction of enteric methane emissions from dairy cattle
Aids in digestion
Digestive aid contains essential trace metals, vitamins, minerals, and other nutrients
Reduces animal methane footprint without altering the production process
Low-methane beef
Low-methane milk

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about.” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

1. A feed product comprising:
from about 10 wt. % to about 95 wt. % of red seaweed material; and
from about 6 wt. % to about 40 wt. % of total oil;
wherein the feed product comprises about 10 wt. % or less of water, based on the total weight of the feed product.

2. The feed product of claim 1, wherein the seaweed material comprises at least one of Asparagopsis taxiformis and Asparagopsis armata.

3. The feed product of claim 1, comprising from about 22 wt. % to about 40 wt. % of total oil, based on the total weight of the feed product.

4. The feed product of claim 1, comprising about 25 wt. % or greater of the seaweed material, based on the total weight of the feed product.

5. The feed product of claim 1, wherein the feed product comprises seaweed material particles and the particles comprise agglomerations of seaweed material and added oil.

6. The feed product of claim 1, wherein the feed product comprises particles and about 90 vol. % or greater of the particles have a diameter of from about 10 μm or greater, based on the total volume of all particles of the feed product.

7. The feed product of claim 1, further comprising molasses, a flow control agent, and/or a binding agent.

8. The feed product of claim 1, further comprising one or more quality extenders.

9. The feed product of claim 1, comprising about 5 wt. % or less of water, based on the total weight of the feed product.

10. The feed product of claim 1, wherein the feed product has a water activity at 25° C. of about 0.6 or less.

11. The feed product of, wherein the seaweed material is freeze-dried.

12. The feed product of, comprising from about 0.2 wt. % to 2 wt. % of one or more halogenated compounds, based on the total weight of the feed product.

13. A method for feeding a feed product of claim 1 to a ruminant, the method comprising administering an amount of the feed product to the ruminant effective to reduce methane emissions in the ruminant.

14. The method of claim 13, further comprising mixing the feed product and a ruminant feed in a mixing device, prior to administering the feed product to the ruminant.

15. A method of making a feed product, the method comprising:
drying red seaweed to form a seaweed material;
converting the seaweed material to particles; and
combining the particles of seaweed material with about 1 wt. % to about 35 wt. % of at least one added oil, thereby producing the feed product, wherein the feed product comprises about 10 wt. % or less of water, based on the total weight of the feed product.

16. The feed product of claim 5, wherein the added oil comprises one or more of canola oil, soybean oil, grape seed oil, coconut oil, palm oil, rapeseed oil, sunflower seed oil, peanut oil, cottonseed oil, palm oil, or olive oil.

17. The feed product of claim 7, further comprising a flow control agent.

18. The feed product of claim 7, further comprising a binding agent.

19. The feed product of claim 1, wherein the seaweed material is freeze-dried.

20. The method of claim 15, wherein drying the seaweed to form the seaweed material comprises freeze-drying.
